(19) United States
(12) Patent Application Publication
(10) Pub. No.: US 2003/0130216 A1
(43) Pub. Date: Jul. 10, 2003

(54) Use of inhibitors of caspase-3 or caspase-activated desoxyribonuclease(cad) for treating cardiac disease

(76) Inventors: Karl-Ludwig Laugwitz, Munchen (DE); Alessandra Moretti, Munchen (DE); Martin Ungerer, Munchen (DE)

Correspondence Address: MYERS BIGEL SIBLEY & SAJOVEC, PO BOX 37428, RALEIGH, NC, 27627, US

(21) Appl. No.: 10/203,333

(22) PCT Filed: Feb. 16, 2001
(86) PCT No.: PCT/DE01/00616

(30) Foreign Application Priority Data
Feb. 16, 2000 (DE) 100 06 889.8

(51) Int. Cl. A61K 48/00

(52) U.S. Cl. 514/44; 424/93.2

(57) ABSTRACT

The invention relates to the use of an inhibitor of caspase-3 or caspase-activated desoxyribonuclease (CAD) for preventing or treating cardiac disease, especially insufficiency of the left ventricle. According to a particular embodiment of the invention, the inhibitor is ICAD. The inhibitor is preferably administered through an adenovirus vector containing the gene that codes for this inhibitor in an expressible form. The invention also relates to methods for identifying inhibitors for the inventive therapeutic application, i.e. compounds which inhibit caspase-3 or CAD or the expression of genes that code for these compounds.

The present invention relates to the use of an inhibitor of caspase-3 or caspase-activated deoxyribonuclease (CAD) for the prevention or treatment of cardiac diseases, the cardiac diseases being especially insufficiency of the left ventricle. In a particular embodiment of the present invention, the inhibitor is ICAD. The administration of the inhibitor is effected especially through an adenovirus vector that contains the gene coding for the inhibitor in an expressible form. Finally, the present invention relates also to processes for identifying inhibitors for the above therapeutic application, that is to say compounds that inhibit caspase-3 or CAD or the expression of the genes coding for those compounds.

Apoptosis is a genetically controlled form of cell death which is essential for the normal development and the physiological equilibrium of organisms. Many degenerative diseases are associated with abnormally high levels of apoptosis. It has been possible to identify the programmed cell death of the cardiomyocytes in a number of cardiovascular diseases, for example in myocardial infarction and congestive cardiac insufficiency (congestive heart failure, CHF). Apoptosis could be the result of prolonged growth stimulation of adult cardiomyocytes which—as terminally differentiated tissue—are no longer capable of division. As compensation for the chronically changed haemodynamic requirements in respect of the failing cardiac muscle, a hypertrophic reaction occurs, and the latter is mediated by the systemic and local upward regulation of the adrenergic system and of the renin/angiotensin system and by various cytokines. However, no therapies for the above-mentioned cardiac diseases have hitherto been used which aimed to inhibit apoptosis. Pharmacological interventions carried out hitherto in cases of such cardiac diseases, which interventions are based predominantly on β-adrenergic blocking, are not successful in all cases or often exhibit an inadequate action.

Accordingly, the invention is based substantially on the technical problem of providing alternative means which are of use in the pharmacological therapy of cardiac diseases.

That technical problem has been solved by the provision of the embodiments characterised in the patent claims. Apoptosis of the cardiac muscle is a cell suicide machine which is regulated in a highly complex manner and in which two main signal pathways lead to activation of the caspase family and to promotion of the translocation of caspase-activated DNase (CAD) into the nucleus: ( a) “death receptor” signalling (e.g. Fas, TNF and DR3-DR6 receptors) and (b) release of cytochrome b from the mitochondria and subsequent transactivation of procaspase 9 by Apaf. The caspase family of the cysteine proteases regulates the onset of apoptosis in mammals. Caspase-3 is the key enzyme and it cleaves targets located upstream which are involved in the expression of the apoptotic phenotype, e.g. gelsolin, PAK2, nuclear lamins and, especially, the inhibiting subunit of the DNA fragmentation factor (ICAD). The most important biochemical feature of apoptosis is the cleavage of chromosomal DNA into nucleosomal units, which appears to be the final event in apoptosis. The nuclease responsible for DNA degradation in apoptosis is CAD. CAD is produced as a complex with ICAD for inhibition of its DNase activity. The caspase-3 activated upstream by apoptotic stimuli cleaves ICAD, which then allows CAD to enter the nucleus and degrade chromosomal DNA. During the investigations which led to the present invention, it was found that the activities of caspase-3 and CAD, the two key molecules in the process of myocardial apoptosis, are increased in an animal model of CHF, in which the changes on the haemodynamic and biochemical level are the same as those in the corresponding disease of the human heart. The stimulation of caspase-3 leads ultimately to the activation of CAD, and both enzymes are therefore required for cardiac insufficiency to progress. It has been possible to show in the investigations that caspase-3 and CAD activity could be inhibited significantly by adenovirus-mediated expression of p35 or ICAD in cardiomyocytes. The inhibition of those key enzymes was also mirrored by reduced DNA fragmentation by ICAD and, although to a lesser extent, by p35. Since ICAD expression was clearly more effective for inhibiting DNA fragmentation in vitro, expression of the transgene coding for ICAD was also studied in vivo in rabbits with cardiac insufficiency. While the infection of rabbit myocardium with recombinant control adenoviruses had no effect on cardiomyocyte apoptosis, the haemodynamic function of ICAD-expressing hearts with insufficiency was at least partially restored (FIG. 6). It was thus possible to observe an improved contractibility and a reduced end-diastolic pressure of the left ventricle with a reduction of caspase-3-induced DNA fragmentation. It is assumed that, in the above syndromes, most of the cardiomyocytes are in a preapoptotic state with increased activities of enzymes involved in apoptosis, which expresses a readiness of those cells for apoptosis, but does not yet indicate that that process has actually taken place. The present results show, however, that myocardial apoptosis contributes to the progression of cardiac insufficiency and that the prevention of apoptosis in cardiomyocytes has a useful function, that is to say the prevention of apoptosis is clearly an attractive goal for therapeutic intervention. By means of the results with insufficient hearts infected with Ad-p35 or Ad-ICAD, it has also been possible to show that an anti-apoptotic approach in the case of cardiac insufficiency not only prevents nuclear DNA fragmentation, but also maintains sarcomere organisation and hence improves the contractile force of the cardiac muscle cells that are still living. Accordingly, for therapeutic intervention in the cardiac diseases discussed above, which are associated with apoptosis of cardiomyocytes, the administration of a factor that inhibits the activity of CAD or caspase-3, or of the gene coding therefor, may be of benefit. On the other hand, the possibility of inhibiting the expression of the gene coding for CAD or caspase-3 may also be therapeutically useful. Such an inhibition may, therefore, take place at various levels, for example at genetic level (“knock out”, inhibition of translation by antisense RNAs or ribozymes) or at protein level (by way of CAD or caspase-3 inhibiting antibodies, ICAD, etc.).

Accordingly, the present invention relates to the use of an inhibitor of caspase-3 or caspase-activated deoxyribonuclease (CAD) for the prevention or treatment of cardiac diseases, especially of cardiac insufficiency, especially insufficiency of the left ventricle.

In a preferred embodiment of the present invention, the inhibitor is a compound that inhibits expression of the gene coding for CAD or caspase-3, for example a ribozyme or an antisense RNA. Since the entire nucleic acid sequence of the gene coding for CAD or caspase-3 is known, the person skilled in the art is able to identify such compounds by routine processes and test their action, for example by means of the procedures described in the Examples below.

A more greatly preferred embodiment of the present invention therefore relates to an antisense RNA which is characterised in that it is complementary to the mRNA transcribed by the gene coding for CAD or caspase-3, or to a portion thereof, preferably the coding region, and is able to bind specifically to that mRNA, as a result of which the synthesis of CAD or caspase-3 is reduced or inhibited. A further more greatly preferred embodiment of the present invention relates to a ribozyme which is characterised in that it is complementary to the mRNA transcribed by the gene coding for CAD or caspase-3, or to a portion thereof, and is able to bind. specifically to that mRNA and to cleave it, as a result of which the synthesis of CAD or caspase-3 is reduced or inhibited. Starting from the disclosed CAD or caspase-3 sequences, the person skilled in the art is able to prepare and use suitable antisense RNAs. Suitable procedures are described in EP-B1 0 223 399 or EP-A1 0 458, for example. Ribozymes are RNA enzymes and consist of a single RNA strand. They are able to cleave other RNAs intermolecularly, for example the mRNAs transcribed by the sequences coding for CAD or caspase-3. Such ribozymes must in principle have two domains, (1) a catalytic domain and (2) a domain that is complementary to the target RNA and is able to bind thereto, which is the prerequisite for cleavage of the target RNA. Starting from procedures described in the literature, it is in the meantime possible to construct specific ribozymes that cut a desired RNA at a particular, pre-selected site (see, for example, Tanner et al., in: Antisense Research and Applications, CRC Press, Inc. (1993), 415-426).

In a further preferred embodiment of the use according to the invention of caspase-3 or CAD inhibitors, the inhibitor is the ICAD or Baculovirus-p35 described in the Examples below.

In a further preferred embodiment of the use according to the invention of caspase-3 or CAP inhibitors, the inhibitor is an antibody that binds to caspase-3 or CAP, or a fragment thereof. Such antibodies may be monoclonal, polyclonal or synthetic antibodies or fragments thereof. In this connection, the term “fragment” means all parts of the monoclonal antibody (e.g. Fab, Fv or single chain Fv fragments) that have the same epitope specificity as the complete antibody. The preparation of such fragments is known to the person skilled in the art. The antibodies according to the invention are preferably monoclonal antibodies. The antibodies according to the invention can be prepared according to standard processes, wherein the caspase-3 or CAP protein or a synthetic fragment thereof preferably serves as the immunogen. Processes for obtaining monoclonal antibodies are known to the person skilled in the art. The monoclonal antibody may be an antibody originating from an animal (e.g. mouse), a humanised antibody or a chimeric antibody or a fragment thereof. Chimeric antibodies resembling human antibodies or humanised antibodies possess a reduced potential antigenity, but their affinity in respect of the target is not reduced. The preparation of chimeric and humanised antibodies, or of antibodies resembling human antibodies, has been comprehensively described (see, for example, Queen et al., Proc. Natl. Acad. Sci. USA 86 (1989), 10029, and Verhoeyan et al., Science 239 (1988), 1534). Humanised immunoglobulins have variable basic structure regions, which originate substantially from a human immunoglobulin (referred to as the acceptor immunoglobulin) and the complementarity of the determining regions, which originate substantially from a non-human immunoglobulin (e.g. from the mouse) (referred to as the donor immunoglobulin). The constant region(s), where present, also originate(s) substantially from a human immunoglobulin. When administering to human patients, humanised (as well as human) antibodies offer a number of advantages over antibodies from mice or other species: (a) the human immune system should not recognise the basic structure or the constant region of the humanised antibody as foreign, and the antibody response to such an injected antibody should therefore be less than the response to a completely foreign mouse antibody or a partially foreign chimeric antibody; (b) since the effector region of the humanised antibody is human, it interacts better with other parts of the human immune system, and (c) injected humanised antibodies have a half-life that is substantially equivalent to that of naturally occurring human antibodies, which allows smaller and less frequent doses to be administered in comparison with antibodies of other species.

The inhibitors discussed above are preferably not themselves administered but are administered by means of gene therapy, that is to say the DNA sequences coding for those inhibitors (e.g. ribozymes, antisense RNAS, antibodies, ICAD, Baculovirus-p35), preferably inserted in an expression vector, are brought to the target organ. Accordingly, the present invention also includes expression vectors containing DNA sequences coding for those inhibitors. The term “vector” refers to a plasmid (pUC18, pBR322, pBlueScript, etc.), to a virus or to another suitable vehicle. The DNA sequences are functionally linked in the vector to regulatory elements which permit their expression in prokaryotic or eukaryotic host cells. In addition to the regulatory elements, such vectors contain, for example, a promoter, typically an origin of replication and specific genes which permit the phenotypical selection of a transformed host cell. The regulatory elements for expression in prokaryotes, for example E. coli, include the lac-,trp promoter or T7 promoter, and for expression in eukaryotes the AOX1 or GAL1 promoter in yeast and the CMV, SV40-, RVS-40 promoter, CMV or SV40 enhancer for expression in animal cells. Suitable regulatory sequences are additionally described in Goeddel: Gene Expression Technology: Methods in Enzymology 185, Academic Press, San Diego, Calif. (1990). Further examples of suitable promoters are the metallothionein I and the polyhedrin promoter. Suitable expression vectors for E. coli include, for example, pGEMEX, pUC derivatives, pGEX-2T, pET3b and pQE-8. The vectors suitable for expression in yeast include pY100 and Ycpad1, for expression in mammalian cells pMSXND, pKCR, pEFBOS, cDM8 and pCEV4 as well as vectors originating from pcDNAI/amp, pcDNAI/neo, pRc/CMV, pSV2gpt, pSV2neo, pSV2-dhfr, pTk2, pRSVneo, pMSG, pSVT7, pko-neo and pHyg.

The DNA sequences described above are preferably inserted into a vector suitable for gene therapy, for example under the control of a tissue-specific promoter, and transferred to the cells. In a preferred embodiment, the vector containing the above-described DNA sequences is a virus, for example an adenovirus, Vaccinia virus or retrovirus. Examples of suitable retroviruses are MoMuLV, HaMuSV, MuMTV, RSV or GaLV. Adenoviruses are particularly preferred, especially those having E1 and/or E3 mutations (deletions), which may additionally also have an E4 mutation (deletion), or “gutless” adenoviruses. Vectors suitable for gene therapy are additionally disclosed in WO 93/04701, WO 92/22635, WO 92/20316, WO 92/19749 and WO 92/06180. For the purposes of gene therapy, the DNA sequences according to the invention may also be transported to the target cells in the form of colloidal dispersions. These include, for example, liposomes or lipoplexes (Mannino et al., Biotechniques 6 (1988), 682).

General processes known in the field can be used for the construction of expression vectors which contain those DNA sequences and suitable control sequences. Such processes include, for example, in vitro recombination techniques, synthetic processes, as well as in vivo recombination techniques, as are described in common textbooks.

The above compounds, or the DNA sequences or vectors coding therefor, are optionally administered together with a pharmaceutically acceptable carrier. Suitable carriers and the formulation of such medicaments are known to the person skilled in the art. Suitable carriers include, for example, phosphate-buffered sodium chloride solutions, water, emulsions, for example oil/water emulsions, wetting agents, sterile solutions, etc.. The suitable dosage is determined by the doctor providing the treatment and is dependent on various factors, for example on the age, the sex and the weight of the patient, on the nature and stage of the cardiac disease, on the nature of the administration, etc..

Finally, the present invention relates also to a process for identifying a compound that is effective as an inhibitor in the above-described therapeutic procedures, the process comprising (a) bringing the test compound that is possibly suitable as an inhibitor into contact with caspase-3 or CAD or with the gene coding for caspase-3 or CAD, and (b) determining the residual caspase-3 or CAD activity or the lowering of gene expression, preferably in comparison with a control assay in which the test compound is not present. A lowered or completely inhibited enzyme activity or gene expression indicates that the test compound is effective as an inhibitor. Suitable assays are known to the person skilled in the art and are also described, for example, in the examples below. The process can also be carried out in a cellular assay. The test compounds may be a wide variety of compounds, both naturally occurring compounds and synthetic, organic and inorganic compounds, as well as polymers (e.g. oligopeptides, polypeptides, oligonucleotides and polynucleotides) as well as small molecules, antibodies, sugars, fatty acids, nucleotides and nucleotide analogues, analogues of naturally occurring structures (e.g. peptide “imitators”, nucleic acid analogues, etc.) and numerous other compounds. In addition, a large number of possibly useful compounds that inhibit cardiomyocyte apoptosis can be screened in natural product extracts as starting material. Such extracts may originate from a large number of sources, for example of the kind fungi, actinomycetes, algae, insects, protozoa, plants and bacteria. The extracts that exhibit activity can then be analysed in order to isolate the active molecule. See, for example, Turner, J. Ethnopharmacol. 51 (1-3) (1996), 39-43 and Suh, Anticancer Res. 15 (1995) 233-239. Fundamentally suitable assay formats for the identification of test compounds that affect the expression or activity of CAD or caspase-3 are well known in the biotechnology and pharmaceutical industry, and additional assays and variations of such assays are obvious to the person skilled in the art. Changes in the level of expression of caspase-3 or CAD can be investigated using processes well known to the person skilled in the art. These include monitoring of the mRNA concentration (e.g. using suitable probes or primers), immunoassays in respect of the protein concentration, RNAse protection assays, amplification-based assays or any other means suitable for detection that is known in the field.

The search for compounds that are effective for therapy by prevention of cardiomyocyte apoptosis can also be carried out on a large scale, for example by screening a very large number of possible compounds in substance libraries, it being possible for the substance libraries to contain synthetic or natural molecules. In any case, the preparation and the simultaneous screening of large banks of synthetic molecules can be carried out by means of well known processes of combinatory chemistry, see, for example, van Breemen, Anal. Chem. 69 (1997), 2159-2164 and Lam, Anticancer Drug Des. 12 (1997), 145-167. The process according to the invention can also be greatly accelerated as high throughput screening. The assays described herein can be suitably modified for use in such a process. It is obvious to the person skilled in the art that numerous processes are available for that purpose.

### BRIEF DESCRIPTION OF THE FIGURES

FIG. 1: Haemodynamic and echocardiographic characterisation of the cardiac insufficiency model

Transthoracic M-mode echocardiographic recordings in control animals (top) and CHF animals (bottom). The diameters of the left ventricle are indicated by a double-headed arrow; EDD: end-diastolic diameter; ESD: end-systolic diameter. After two weeks, the animals with a pacemaker exhibit ventricle dilatation with reduced wall movements, which indicates reduced cardiac function and increased wall stress.

FIG. 2: Induction of caspase-3-mediated CAD activation and subsequent DNA fragmentation in isolated ventricular cardiomyocytes of control myocardium and insufficient myocardium (CHF)

a: Fluorogenic assay of the caspase-3 enzyme levels measured in cytosolic fractions of isolated myocytes of control myocardium and myocardium with pacemaker (CHF); n=5 per group, p<0.005 ANOVA with Scheffé post-hoc analysis.

b: Fragmentation of genomic DNA which had been isolated from isolated rabbit liver nuclei and was incubated with cytosolic extracts of control myocardium and insufficient cardiomyocytes (CHF). The DNA fragmentation mirrors the activity of the caspase-activated deoxyribonuclease (CAD).

c: Isolated DNA from the heart of insufficient animals after 7 days (CHF 7) and 15 days (CHF 15) showed a rise in DNA laddering in comparison with control tissue.

d: Freed DNA/histone complex was quantified with an ELISA system of control cardiomyocytes and cardiomyocytes with pacemaker.

The data are expressed as mean values±SEM; n=5 per group; p<0.005.

FIG. 3: Blocking of caspase-3 activity and of DNA fragmentation by adenoviral overexpression of p35 and ICAD in vitro (a,b) and in vivo (c,d)

a: Caspase-3 activity was measured in isolated control cardiomyocytes and TNFα-stimulated cells in the presence of an adenovirus construct for p35 (Ad-p35), of the empty construct (Ad-GFP) and of the tetrapeptide inhibitor for caspase-3 (DEVD).

b: The DNA/histone formation was determined in isolated control cells and TNFα-stimulated ventricular cardiomyocytes after adenovirus infection with Ad-p35, Ad-ICAD and Ad-GFP (as control).

c: Cardiomyocytes were isolated from control myocardium and myocardium with pacemakers (CHF), and the caspase-3 activity was measured in cytosolic extracts. The 3rd and 4th columns represent enzyme activity measurements of cardiomyocytes from myocardium of the left ventricle after adenovirus gene transfer of Ad-p35 and Ad-GFP (control).

d: Ventricular myocytes were isolated from control hearts and hearts with pacemakers (CHF) and the DNA/histone formation was quantified in cell-free extracts. The 3rd and 4th columns represent the DNA fragmentation analysis on cells of animals after myocardial gene transfer with Ad-ICAD and the control adenovirus Ad-GFP.

e: Immunoblot analyses of cytosolic extracts from the anterolateral wall of myocardium infected with Ad-p35 0, 4, 7 and 15 days after gene transfer. The immunostaining was carried out with the two monoclonal antibodies anti-Flag M2 and anti-α-sarcomere actin antibody for p35, in order to allow documentation of the expression of p35 and actin in parallel.

The data are expressed as mean values±SEM; n=4 per group; p<0.01, p<0.001.

FIG. 4: Adenovirus gene transfer to the failing myocardium

a: Macroscopic sections of rabbit hearts, in which an adenovirus coding for β-galactosidase had been injected under ultrasound control (section thickness: 7 μm, distance between individual sections: 200 μm). The sections were stained with X-Gal. The left ventricular myocardium is designated LV, the right ventricular myocardium is designated RV.

b: Immunoblot analysis of cytosolic extracts of myocardium infected with Ad-GFP, Ad-ICAD and Ad-p35.

c: Fragmentation of genomic DNA which had been isolated from isolated rabbit liver nuclei, on incubation with cytosolic extracts of isolated ventricular control cardiomyocytes and insufficient myocytes (CHF). In addition, the activity of the DNA fragmentation of cardiomyocytes after adenovirus gene transfer with Ad-ICAD and Ad-GFP was analysed in vivo.

FIG. 5: Tissue sections under light after in vivo gene transfer with the adenovirus constructs for p35 and ICAD

a: The GFP expression in macroscopic sections of infected myocardium was visualised by phase-contrast fluorescence microscopy using a 450-490 nm filter.

b: The direct transgene expression after Ad-ICAD infection was demonstrated by immunostaining with a monoclonal anti-FLAG antibody against the synthetic epitope introduced into both transgenes. The sample was visualised by fluorescence microscopy using a 546 nm filter (rhodamine fluorescence).

c,d: Ventricular cardiomyocytes were isolated from myocardium infected with Ad-p35, and the transgene expression was documented by fluorescence microscopy for GFP and the anti-FLAG antibody.

FIG. 6: Echocardiographic and haemodynamic measurements on hearts treated with Ad-ICAD

Cardiac catheterisation was carried out in the basal state and under increasing adrenalin concentrations.

a: fractional shortening recordings on control hearts and hearts infected with Ad-GFP or Ad-ICAD after 15 days with pacemaker provision. The percentage of fractional shortening was calculated as % FS=[(EDD-ESD)/EDD]×100.

b: The end-diastolic diameters of the left ventricle are shown for the control and for myocardium infected with Ad-GFP or Ad-ICAD, after a pacemaker time of 15 days.

c: The end-diastolic pressures of the left ventricle were determined for the control and for Ad-GFP or Ad-ICAP animals after pacemaker provision.

d: The development of LVEDP during the period of pacemaker provision is shown for Ad-GFP, Ad-ICAD and control animals after 7 and 15 days.

e: dp/dtmax recordings of control animals and animals treated with Ad-ICAD, after administration of increasing concentrations of adrenalin. The data are shown as mean values±SEM.

FIG. 7: Echocardiographic and haemodynamic measurements on hearts treated with Ad-p35

Infection with the adenovirus constructs was carried out before the start of the pacemaker period.

a: Fractional shortening was estimated in the control, CHF and at 15 days after the transcoronary administration of Ad-GFP or Ad-p35 to hearts provided with a pacemaker.

b: Course of fractional shortening with time after 7 and 15 days' pacemaker provision.

c: The reduced end-diastolic ventricular pressure (LVEDP) was estimated in the control, CHF and at 15 days after the transcoronary administration of Ad-GFP or Ad-p35 to hearts provided with a pacemaker.

d: dp/dtmax recordings after administration of increasing doses of epinephrine. The data are shown as mean values± SEM (n=8 per group; * p<0.05; ** p<0.001) in comparison with the control and CHF and Ad-GFP hearts.

FIG. 8: Effect of p35 expression on the sarcomere organisation and the contractile force of muscle cells of hearts provided with a pacemaker

a,b,c: Ventricular rabbit cardiac muscle cells were isolated from the anterolateral wall of the control (a), CHF (b) and Ad-p35-infected insufficient myocardium (c) and visualised after phalloidin staining by means of confocal laser scanning microscopy.

d: Contraction amplitude in isolated cardiac muscle cells (n=60 cells of four rabbits per group).

e: The morphology of phalloidin-stained muscle fibres was evaluated semi-quantitatively on the basis of the area occupied by organised sarcomere in the total cell region: weak, less than ⅓ of the cell region (black region); moderate, less than ⅔ of the cell region (grey region); good, total cell region (empty region). 120 cells isolated from 4 animals were counted for each group. Data (in d) were expressed as the mean value±SEM (* p<0.001 in comparison with myocardium provided with a pacemaker (CHF+Ad-GFP)).

FIG. 9: Microinjection of activated caspase-3 into the cytoplasm of healthy ventricular cardiac muscle cells

a,b: FITC-conjugated dextran alone (a) or FITC-conjugated dextran+human recombinant active caspase-3 (b) (left-hand image: 4 nm/μM; right-hand image: 20 ng/μl) was injected into muscle cells. The morphology of the actin fibres was visualised by means of confocal laser scanning microscopy after phalloidin staining.

c: The contraction amplitude under basal conditions and isoprenaline stimulation (10⁻⁸ mol/l) was determined in the cells after microinjection of FITC-conjugated dextran (as control) or caspase-3 (CPP32). Pre-incubation with DEVD-fmk (R and D Systems, Wiesbaden, Germany) was also carried out (grey column). n=45 cells for each group. The data in c are shown as the mean value±SEM (* p<0.005 in comparison with injected control cells (basal; 10⁻⁸ mol/l isoprenaline).

FIG. 10: Effect of ICAD on the contractility of individual cardiac muscle cells

Contraction amplitude in isolated cardiac muscle cells (n=60 cells from four rabbits per group). Test was carried out as described in FIG. 8d; n.s.=not significant, ** p<0.05.

The following Examples illustrate the invention.

### EXAMPLE 1

### General Process

### (A) Construction and Purification of Recombinant Adenovirus

Recombinant adenoviruses (E1 and E3 deficient; serotype 5) coding for the inhibitor of caspase-activated DNAse (ICAD) of the rat or for the Baculovirus apoptosis suppressor p35 were prepared as described by He et al., Proc. Natl. Acad. Sci. USA, 95, (1988), 2509-2514. To that end, the sequence coding for ICAD or p35 and provided at the N-terminus with the “Flag” epitope (Stratagene, La Jolla, Calif., USA) was inserted into the polylinker sequence of the GFP-expressing “pAdTrack” vector between a tissue-non-specific cytomegalivirus (CMV) promoter and a SV40 polyadenylation signal (Enari et al., Nature 391, (1988), 43-50), (Davidson and Steller, Nature 391, (1998), (587-591), (He et al., supra), (Inan et al., Onkogene 13, (1996) 749-755). The resulting plasmid was then co-transformed together with the “pAdEasy-1” plasmid into electrocompetent BJ5138 bacteria (He et al., supra). For the preparation of a control adenovirus containing only the gene for enhanced GFP, a co-transformation with the plasmids “pAdTrack” and “pADEasy” was carried out. Recombinant adenovirus vector DNA was extracted from positive clones and transfixed into subconfluent HEK 293 cells using a “SuperFect™” transfection reagent (QIAGEN, Hilden, Germany). Recombinant viruses (Ad-ICAD, Ad-p35 and Ad-GFP) were obtained from the cell lysate and were analysed by means of PCR and restriction cleavages.

After the isolation, recombinant adenoviruses were prepared on a large scale by infection of subconfluent HEK 293 cells in 150 mm plates (moi: 5 pfu/cell). 48 to 72 hours after infection after occurrence of the cytopathogenic action, the cells were harvested by scraping off and centrifugation for 20 minutes at 1000×g. The cell pellet was re-suspended in PBS and 0.25% Triton-X 100™. The homogenate was incubated for 10 minutes at room temperature and the nuclei of the opened HEK cells were removed by a centrifugation step at 2500×g (20 minutes). The supernatant containing the virus particles was introduced onto a CsCl step gradient (1.3 and 1.4 g of CsCl/ml, dissolved in TE buffer) and subjected to ultracentrifugation for 1.5 hours at 10° C. and at 150,000×g. The virus band forming in the 1.3 and 1.4 g/ml interface was removed with a syringe, dialysed against PBS containing 1% saccharose and 10% glycerol, and stored in aliquots at −80° C. Adenovirus titres were determined by means of plaque titration on HEK 293 cells (Krown et al., J. Clin. Invest. 98 (1996), 2854-2865).

### (B) Cell Cultivation and Adenovirus Infection of H9c2 Cardiomyoblasts

H9c2 cardiomyoblasts (ATCC CRL 1446, rat cardiomyoblasts) were cultured in monolayers in DMEM, 10% FBS, 2 mmol/l glutamine, penicillin (100 IE/ml) and streptomycin (100 μg/ml) in 10% CO₂ at 37° C. in a humidified incubator. After the cardiomyoblasts had reached 70 to 80% confluence, they were transfected in PBS with a suitable adenovirus titre. After incubation for one hour at room temperature, the culture medium was returned to the plates. 36 to 48 hours after adenovirus infection, the cells were used for the individual experiments.

### (C) Preparation and Culturing of Adult Ventricular Cardiomyocytes of Rats and Rabbits

Individual calcium-tolerant ventricular myocytes were isolated from 12- to 16-week-old Wistar rats (300-450 g) or from 3-month-old male New Zealand rabbits (2.8-3 kg). The excised hearts were perfused via the aorta, at a constant through-flow rate, with Ca²⁺-free “Powell” medium (110 mmol/l NaCl, 2.5 mmol/l KCl, 1.2 mmol/l KH₂PO₄, 1.2 mmol/l MgSO₄, 11-mmol/l glucose, 25 mmol/l NaHCO₃, equilibrated with 5% CO₂ and adjusted to a pH value of 7.4). After 10 minutes, enzymatic digestion was replaced by replacement of the Ca²⁺-free buffer by 110 or 218 IE/ml (for rats or rabbits, respectively) of type II collagenase (Worthington, Freehold, N.J., USA) and “Powell” medium containing 30 or 40 μmol/l Ca²⁺ (for rats or rabbits, respectively). After thorough digestion of the tissue, the heart was removed from the perfusion device, and the ventricular muscle was exposed and dissociated mechanically in “Powell” medium with constant oxygenation. After filtration over a nylon membrane (200 μm mesh size), the cell suspension was centrifuged for 3 minutes at 20×g and the cells were re-suspended in “Powell” medium containing 0.2 mmol/l Ca²⁺. The cells were allowed to settle, and the pellet was collected in “Powell” medium containing 0.4 mmol/l Ca²⁺ and carefully introduced onto a 4% bovine serum albumin (BSA) gradient in “Powell” medium (1 mmol/l Ca²⁺). After centrifugation for 2 minutes at 20×g, the cardiomyocytes were resuspended in M199 culture medium (supplemented with MEM vitamins, MEM non-essential amino acids, 25 mmol/l HEPES, 10 μg/l insulin, 100 IE/ml penicillin, 100 μg/ml streptomycin and 100 μg/ml gentamicin), plated out on laminin-precoated dishes (5-10 μg/cm²) in a density of 10⁵ cells per cm² and cultured in a humidified atmosphere (5% CO₂) at 37° C. Infection of the cardiomyocytes with the adenoviruses took place 6 to 8 hours after the plating out in M199 culture medium. After isolation of the cells of normal or insufficiently in vivo-infected-rabbits and use for the determination of the occurrence of apoptosis, they were directly frozen at −80° C. in the form of a pellet immediately after centrifugation in a 4% BSA gradient.

### (D) Western Blot Analysis

(I) For the immunological determinaton of the induced proteins GFP, ICAD and p35, the H9c2 cardiomyoblasts were harvested 48 hours after infection (moi: 50 pfu/cell) in 10 mM HEPES buffer, pH value 7.0 (which contained 40 mM β-glycerol phosphate, 50 mM NaCl, 2 mM MgCl₂, 5 mM EGTA, 1 mM DTT, 2 mM ATP, 10 mM creatine phosphate, 50 μg/ml creatine kinase, 1 mM PMSF, 1 μg/ml leupeptin, 1 μg/ml pepstatin, 10 μg/ml aprotinin, 16 μg/ml benzamidine, 10 μg/ml phenanthroline) and broken up by four freezing/thawing cycles. The resulting lysates were centrifuged for 30 minutes at 15,000 rpm and the protein concentrations were determined in a “Bradford” assay. Equal protein amounts (50-200 μg) were diluted with SDS application buffer, separated on a 12% polyacrylamide gel, and transferred by electrophoresis to a nitrocellulose membrane (Bio-Rad Laboratories, Munich, Germany). The blots were stained with “Ponceau” red in order to check the protein transfer. After blocking overnight with 5% fat-free milk powder in TBST (10 mM tris-HCl, pH value 8.0, 150 mM NaCl, 0.05 Tween-20™), the membranes were incubated for one hour with anti-FLAG M2 antibodies (Stratagene), which was diluted 1:10,000 (ICAD determination) or 1:1000 (p35 determination) in TBST (with 0.1% BSA and 0.02% Na azide). Antigen/antibody complexes were visualised, by means of chemiluminescence (“ECL” detection kit, Amersham Pharmacia Biotech, Vienna, Austria), after incubation of the membranes for one hour with anti-mouse IgG diluted 1:10,000 and conjugated with horseradish peroxidase (Sigma-Aldrich Chemie GmbH, Munich, Germany).

(II) Native ICAD cleavage products in lysates of sham-operated and failing myocardium were demonstrated using polyclonal goat antibodies against the N-terminus of mouse ICAD (St. Cruz Biotechnology, Santa Cruz, U.S.A.) (dilution: 1:1000). Some extracts of control hearts were also incubated for 30 minutes at 37° C. with 1 ng/μl of recombinant human active caspase-3 in the presence or absence of the tetrapeptide caspase-3 inhibitor DEVD-fmk (R and D Systems, Wiesbaden, Germany) (100 μmol/l). The immunological demonstration of p35 and α-sarcomere actin in extracts of transcoronary sham-operated myocardium was carried out by means of the monoclonal mouse anti-FLAG M2 antibody (dilution: 1:1000) and a monoclonal anti-α-sarcomere actin antibody (Sigma, Taufkirchen, Germany) (dilution: 1:5000).

### (E) Immunocytochemical and Microscopic Studies

Isolated adult ventricular cardiomyocytes, which had been plated out on microscope cover slips coated with 5 μg/cm² of laminin, were infected with control adenovirus Ad-GFT or with Ad-ICAD or Ad-p35 viruses (moi: 50 pfu/cell) and analysed 48 hours after infection. For the determination of the expression of the transgenes in rabbit hearts infected in vivo, frozen tissue was cut on a microtome for frozen sections into disks having a thickness of from 3 to 4 μm. After flushing three times with PBS, the cells and sections were fixed for 15 minutes with 4% paraformaldehyde in PBS and then permeabilised for 10 or 30 minutes with 0.1% saponin in PBS. In order to avoid non-specific antibody binding, the cardiomyocytes were treated for 30 minutes with 10% FBS in DMEM prior to labelling with the monoclonal mouse anti-FLAG M2 (IgG1) antibody (Stratagene, 10 μg/ml). After incubation with a rhodamine-conjugated anti-mouse IgG (Santa Cruz Biotechnology, Santa Cruz, Calif., USA; 4 μg/ml), the samples were visualised by means of phase-contrast fluorescence microscopy using a 450-490 nm filter (GFP fluorescence) and a 546 nm filter (rhodamine fluorescence) (inverse microscope “Axiovert 25”, Zeiss, Jena, Germany).

### (F) ELISA for Histone-Bonded DNA Fragments

Apoptosis in adult ventricular cardiomyocytes was determined by means of a commercially available, quantitative, nucleosome ELISA directed to DNA and histone and using monoclonal mouse antibody (Roche Diagnostics, Mannheim, Germany). The amount of nucleosomes in the lysate of 2×10³ cells was determined via the peroxidase retained in the immune complex and was evaluated by photometry. Three samples were evaluated in each case, the optical density (OD) being measured at 405 nm. The factor increasing apoptosis was calculated for each experimental group as OD (treatment)/OD (control) after subtraction of the background OD₄₀₅.

### (G) Caspase-3 Activity

The activity of caspase-3 was determined by means of the colorimetric “CPP32” assay kit (Clontech Laboratories GmbH, Heidelberg, Germany) by detection of chromophore p-nitroanilide after cleavage of the labelled substrate Asp-Glu-Val-Asp(DEVD)-p-nitroanilide. To that end, 2×10⁶ adult cardiomyocytes were lysed, and equal amounts of protein were reacted with 50 μmol/l of DEVD-p-nitroanilide for one hour at 37° C. The activity was determined by photometry at 405 nm and the results were calibrated with known concentrations of p-nitroanilide. The units of protease activity were defined as the amount of caspase-3 that is required to produce 1 pmol of p-nitroanilide at 25° C.

### (H) ICAD Activity Assay

ICAD activity was calculated by determining the inhibition of CAD in the fragmentation of DNA from rabbit liver nuclei. The nuclei were prepared as described by Blobel and Potter (Science 154 (1966), 1662) and stored at −80° C.

H9c2 cardiomyoblasts were infected with Ad-GFP or Ad-ICAD (moi: 50 pfu/cell), and 48 hours later the CAD activity was stimulated by treatment of the cells with rat α-TNF (500 E/ml) for 5 hours. 100 μg of protein from crude cell lysates were incubated with 2×10⁶ nuclei in a reaction buffer which consisted of 10 mM HEPES, pH value 7.0, 40 mM β-glycerol phosphate, 50 mM NaCl, 2 mM MgCl₂, 5 mM EGTA, 1 mM DTT, 2 mM ATP, 10 mM creatine phosphate, 50 μg/ml creatine kinase and a mixture of protease inhibitors (1 mM phenylmethylsulfonyl fluoride (PMSF), 10 μg/ml leupeptin, 1 μg/ml pepstatin, 10 μg/ml aprotinin, 16 μg/ml benzamidine, 10 μg/ml phenanthroline). After incubation for 90 minutes at 37° C., the nuclei were obtained by centrifugation at 35,000×g (5 minutes) and then lysed for 30 minutes at 56° C. in 100 mM tris-HCl, pH value 8.5, 5 mM EDTA, 0.2 M NaCl, 0.2% SDS, 1 mg/ml proteinase K. The DNA was then precipitated by addition of the same volume of isopropanol, dissolved in 20 μl of tris-HCl, pH value 8.5 (with 1 mM EDTA and 1 mg/ml RNase A) and incubated for 30 minutes at 37° C. The DNA was analysed by gel electrophoresis (1% agarose in the presence of 0.5 μg/ml ethidium bromide).

### (I) Gene Transfer and Operations on the Animal

Adult male white New Zealand rabbits (2-4 kg) were anaesthetised with midazolam (2 mg/kg) and medetomidine (150 μg/kg), intubated and kept breathing artificially. The left chest wall was opened and two isolated pacemaker cables were attached to the left heart. After this procedure, the animals were extubated. In the control animals, a chest wall incision was carried out with implantation of the pacemaker cables, but the pacemaker was never set going. Twelve hours after this operation, a pacemaker speed of 360 beats/minute was initiated. This was checked at the beginning of the tests and then weekly (in total over a period of 15 days) by means of an ECG. The adenovirus gene transfer into the rabbit cardiac muscle was carried out according to known protocols (Weig et al., Circulation 101, (2000), 1578-1585).

### (J) In Vivo Haemodynamic and Echocardiographic Data

The contractile force of the left ventricle was studied before and 7 and 15 days after the adenovirus gene transfer. The rabbits were anaesthetised as described above. The echocardiographic recordings (M mode) were carried out as described in earlier studies (Gardin et al., Circ. Res. 76 (1995), 907-914). In addition, ECG and blood pressure were monitored continuously. After preparation of the right carotid, a “Millar 2.5 French tip” catheter (Hugo Sachs, Freiburg, Germany) connected to a differentiating device was inserted into the left ventricle. The position of the catheter was checked both by fluoroscopy and by observing the blood pressure wave form. After determination of the basal contractile force and of the pressure of the left ventricle (with the pacemaker switched off), 200 μl of 0.9% NaCl were injected as negative control. After a sufficient equilibration period, adrenalin was injected in concentrations of 0.1 to 0.8 ng. Measurements were taken before the surgical operations and 7 and 15 days after the pacemaker period.

### (K) Statistical Analyses

The data are the mean values+SEM of more than three independent experiments. The data were checked for statistically relevant differences by means of “one-way” variance analysis (ANOVA) and, following that, by means of “Scheffé” post-hoc analysis.

### (L) Microinjection

The microinjection experiments were carried out on freshly isolated cardiac muscle cells of sham-operated rabbits by means of a “Femto-Jet” microinjection device (Eppendorf, Hamburg, Germany). FITC-conjugated dextran (6 mg/ml), alone or in combination with human recombinant active caspase-3 (4 ng/μl and 20 ng/μl) in 5 mmol/l potassium phosphate buffer (pH value 7.4; 100 mmol/l KCl) was injected into the cytoplasm of the cells in culture medium (Pᵢ=1000 hPa, tᵢ=0.1 sec., P_{c}=30 hPa) which had been supplemented with 200 μmol/l BDM (butanedione-monoxime, Sigma, Taufkirchen, Germany), 10 μmol/l verapamil and, optionally, 100 μmol/l DEFD-fmk. After incubation for two hours, contraction measurements or phalloidin staining were carried out. Injected cells were selected by means of FITC fluorescence.

### EXAMPLE 2

### Physiological Effects of Chronic Tachycardia in Rabbits

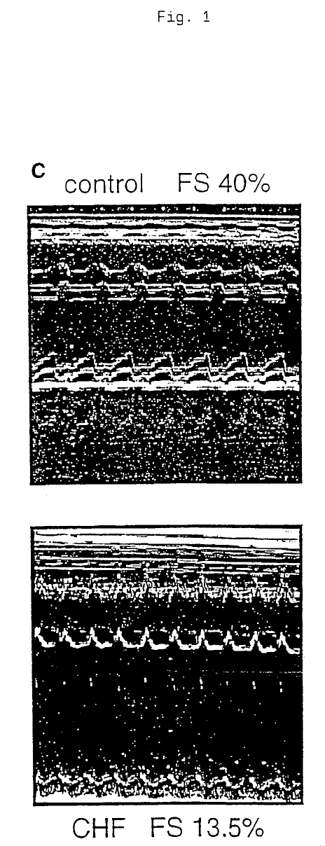

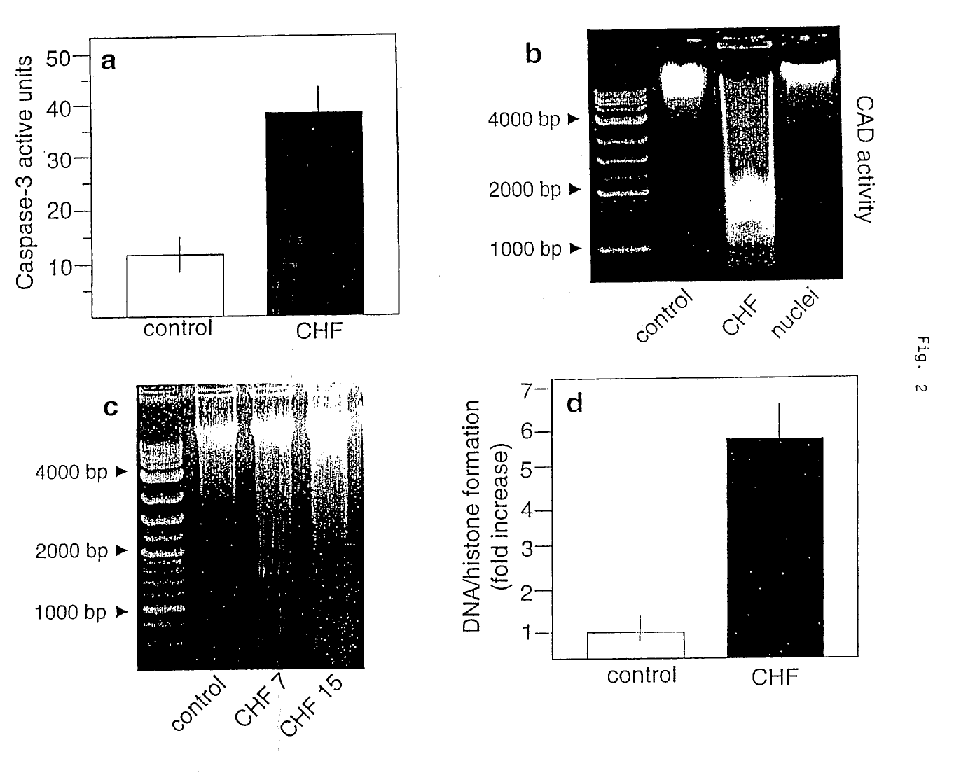

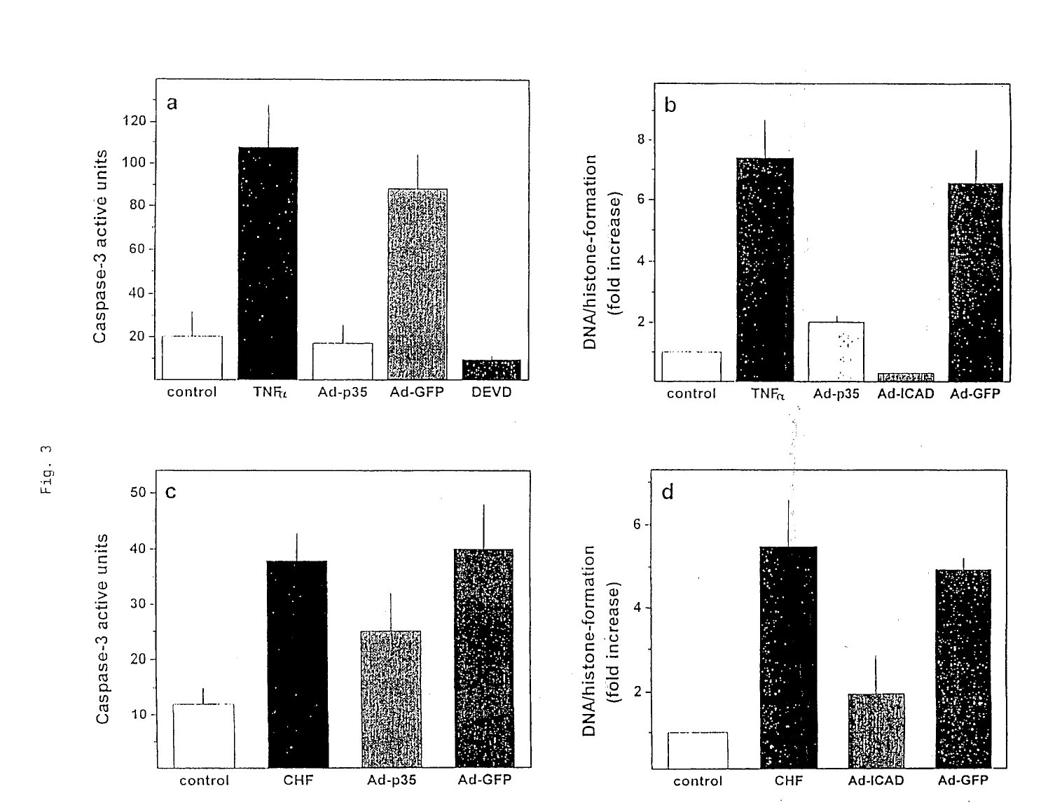

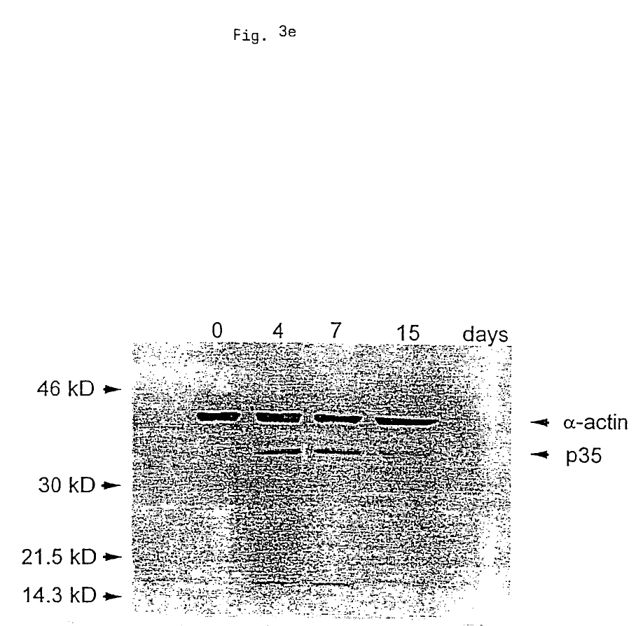

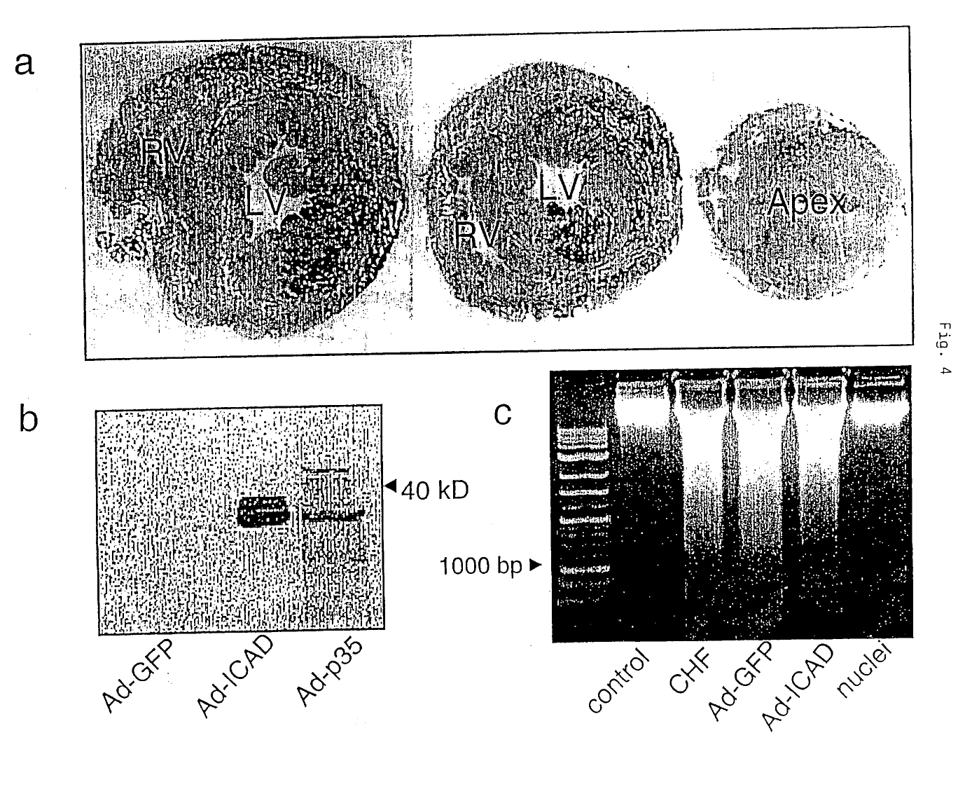

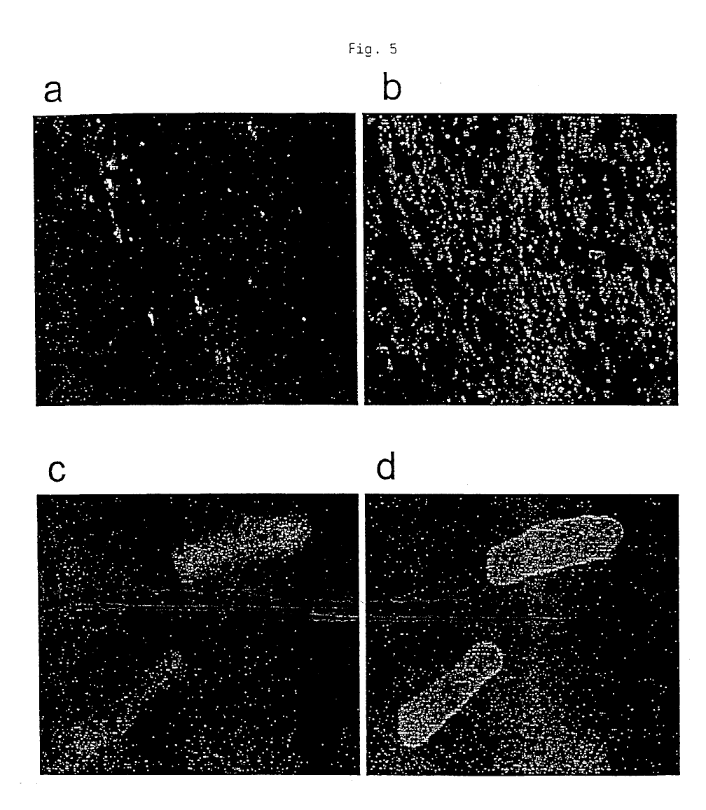

A model of congestive cardiac insufficiency (CHF) with a low minute volume was used, which model corresponds to changes in humans on a haemodynamic and biochemical level. In this test, none of the animals died during surgical instrument implantation, although there was a mortality rate of from 10 to 20% during the 15-day period in which the pacemaker was connected. 15 days' chronic pacemaker provision at 360 beats per minute led to the general clinical finding of systemic cardiac insufficiency including dilatation of both ventricles (FIG. 1c), pleural effusions and abdominal ascites. Echocardiographic studies showed a rise in the extent of the end-diastoles of the left ventricle and a lowering of fractional shortening in the CHF rabbits (Table 1(a)). Haemodynamic measurements likewise showed a higher end-diastolic pressure of the left ventricle and a lower contractile force of the left ventricle (determined by LV+dP/dt) and relaxation (determined by LV−dP/dt) in the rabbits provided with a pacemaker (Table 1(b)). The echocardiographic and haemodynamic measurements were recorded in each rabbit before implantation of the pacemaker and then again after 7 and 15 days (with the pacemaker switched off). Accordingly, each rabbit was used as its own control. Sham-operated rabbits exhibited no difference in respect of haemodynamic parameters. In the myocardium with insufficiency, biochemical changes in the β-adrenergic signal transduction similar to those in the case of cardiac insufficiency in humans were observed. The density of β-adrenergic receptor was significantly reduced in the insufficient myocytes, and the expression of βARK1 was increased.

For control purposes, values prior to implantation of the pacemaker and CHF values three weeks after implantation of the pacemaker were determined. HR, heart rate [beats/min (bpm)]; LVEDD, LV end-diastolic diameter; LVESD, LV end-systolic diameter; FS, fractional shortening, determined as % FS=[(EDD-ESD/EDD]×100; LVEDP, LV end-diastolic pressure; LVSP, LV end-systolic pressure; dp/dtmax, maximum rate of LV pressure increase; dp/dtmin, maximum rate of LV pressure drop.

### EXAMPLE 3

### Apoptosis Parameters in Cardiac Insufficiency

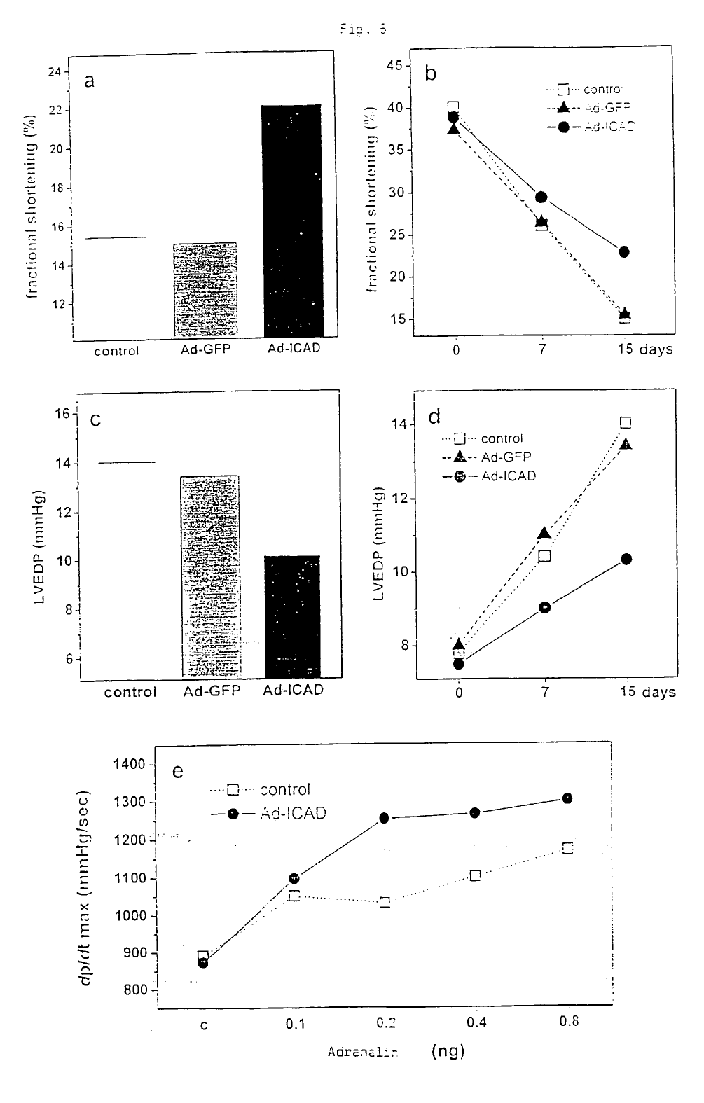

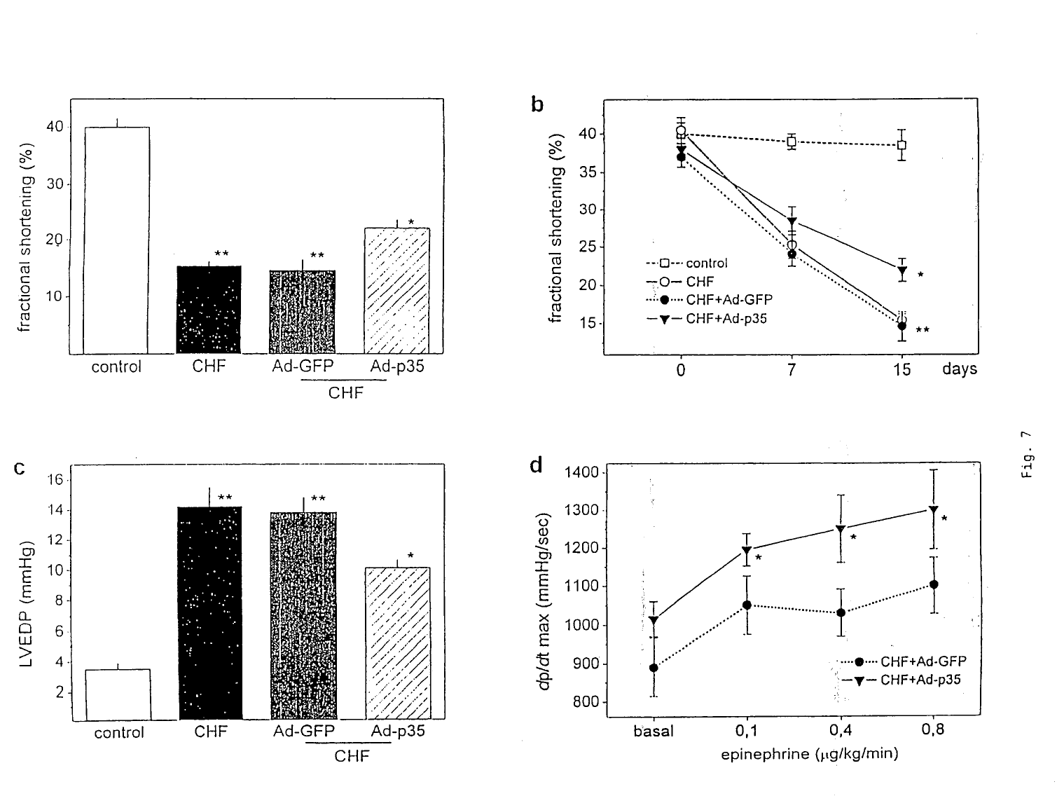

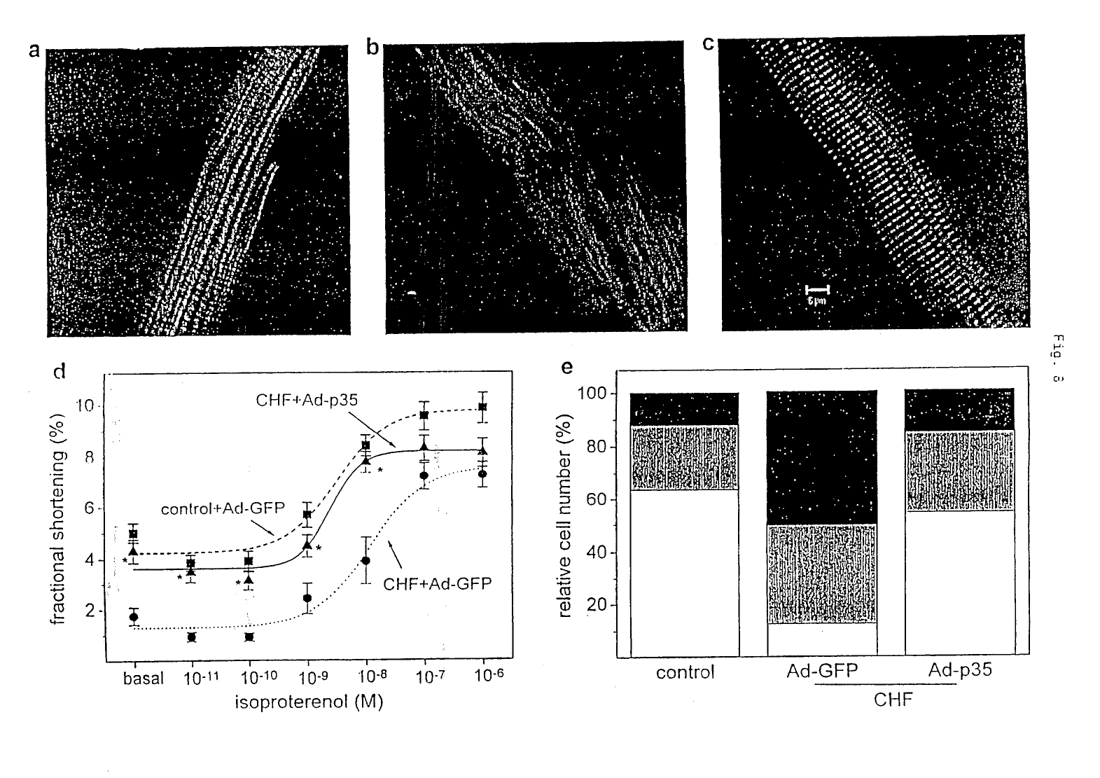

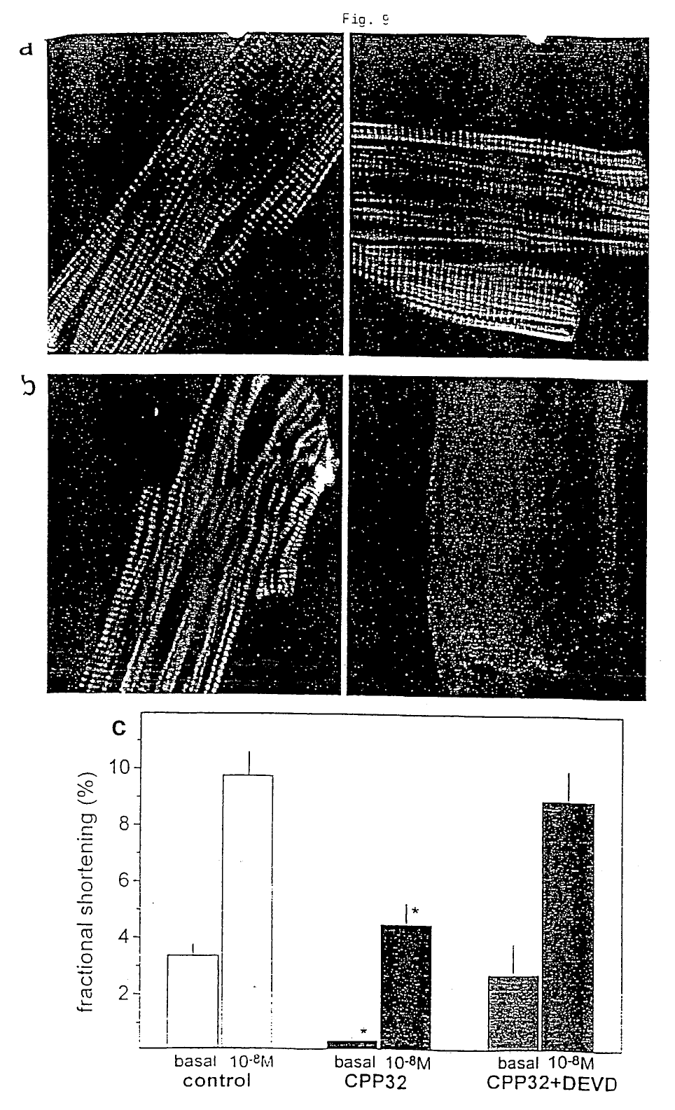

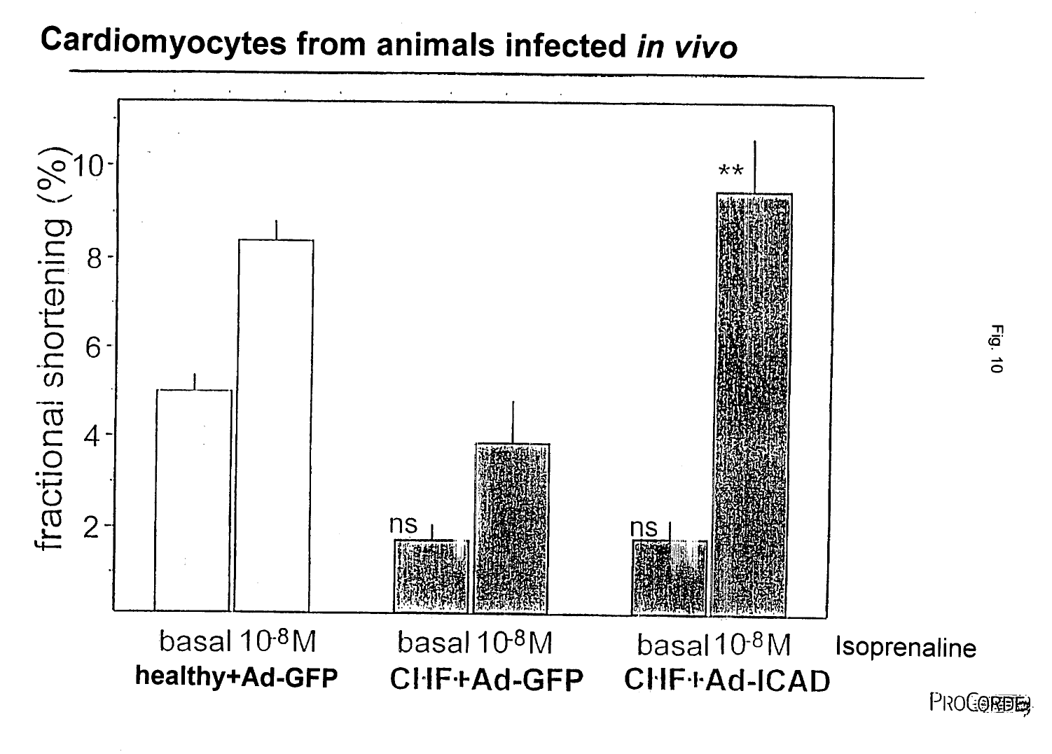

In order to allow evaluation of the most important biochemical features of apoptosis in the hearts of rabbits provided with a pacemaker, adult ventricular myocytes were isolated and a number of molecular analyses were carried out. In the final stage of cardiac failure, apoptosis in the intact myocardium is accompanied by DNA degradation. In order to study the capacity of the cell lysates of insufficient cardiomyocytes to effect DNA degradation in vitro, liver nuclei were incubated with lysates of failing myocytes, and the DNA was studied by means of agarose gel electrophoresis (FIG. 2a). Insufficient myocardium exhibited an increased activity for DNA degradation in comparison with control tissue. It was possible to block that activity by overexpression of ICAD. In addition, in total cell extracts prepared from insufficient and control cardiomyocytes, there was a significant increase (approximately 3-fold) in the caspase-3 activity in the CHF cells (FIG. 2b). Cytosol extracts of the myocardium of animals provided with a pacemaker exhibited an almost 6-fold increase in histone-associated DNA fragments in comparison with extracts from control myocytes (FIG. 2c).

### EXAMPLE 4

### p35 and ICAD Gene Transfer Sufficiently Inhibit Increased Caspase-3 Activity and DNA Fragmentation In Vitro and In Vivo

For the manipulation of the caspase-3-activated DNA degradation signal pathway as the last step in the process of myocardial cell death, adenovirus constructs for p35 as a potent caspase-3 inhibitor (Ad-p35) and ICAD (Ad-ICAD) as a scavenger molecule for activated CAD were produced. The adenovirus constructs were so constructed that they coded for the transgene and, in order to control sufficient expression, in addition for GFP (Ad-GFP). In addition, both transgenes were provided with an epitope “tag” for immunostaining. Before the determination of the functional consequences of the adenovirus infection in insufficient myocardium, the expression of the transgenes in TNFα-stimulated apopototic ventricular myocytes was studied (FIG. 3a,b). Adult cardiomyocytes were infected with adenoviruses having a multiplicity of infection (moi) of 80 pfu/cell, which had already been shown to achieve optimum expression of the transgene in practically 100% of the ventricular myocytes. 48 hours after infection with Ad-p35 or Ad-ICAD, extracts of myocytes expressed significant protein levels, which was determined by immunostaining. The caspase-3 activity and DNA fragmentation were stimulated in vitro by treatment with TNFα (FIG. 3a,b). The adenoviral expression of p35 suppressed the TNFα-stimulated caspase-3 activities to basal level in vitro in ventricuiar rabbit cardiomyocytes. Interestingly, the overexpression of ICAD gave a DNA/histone formation below the control values (FIGS. 3a,b). The infection of cardiomyocytes with recombinant control adenovirus (Ad-GFP) had no effect in vitro and in vivo on the apoptosis parameters in the myocytes (FIGS. 3a,b,c,d). As shown in FIG. 3 and c, isolated myocytes of insufficient myocardium exhibited a significant increase in caspase-3-mediated induction of DNA fragmentation as compared with healthy myocytes (38±6 active units versus 12±3 active units/5.5-fold increase in DNA/histone formation, n=5 in each group, p<0.005). After administration of the two adenovirus transgenes in vivo and subsequent isolation of the ventricular myocytes from the myocardial target region, almost 50% of the cells reproducibly exhibited a positive GFP staining. In those cells, ICAD effectively blocked caspase-3-induced CAD activity, p35 partially inhibited the enzyme activity. Control infections showed no effect (FIGS. 3c,d).

### EXAMPLE 5

### Adenovirus Gene Transfer into the Cardiac Muscle with Insufficiency is Effective

Tissue sections of a representative rabbit heart three days after gene transfer of Ad-βGal (10⁹-10¹⁰ pfu) are shown in FIG. 4a. The gene transfer exhibited maximum gene expression 6 days after infection, which was followed by a gradual fall in expression in the following weeks. After four weeks, less than 1% of the myocardium was infected. Two thirds of the myocardium of the left ventricle reproducibly exhibited expression of the transgene (FIG. 4a). Fluorescence images of macroscopic sections of a heart infected with bicistronic Ad-ICAD showed a marked transgene expression over the entire manipulated myocardium and the immunostaining of the epitope “tag” of the transgene showed signals in the same region (FIGS. 5a,b). After infection with 10⁹-10¹⁰ pfu Ad-ICAD in vivo, almost 50% of the isolated ventricular cardiomyocytes exhibited green fluorescence and a positive immunostaining (FIGS. 5c,d). p35 (as a Baculovirus protein) exhibited lower expression levels than ICAD at viral titres adjusted in exactly the same way in the eukaryotic cells, which is shown by the immunoblot of FIG. 4b.

### EXAMPLE 6

### Blocking of the Caspase-3-Activated Apoptosis Mechanism Improves the Contractile Force

In order to assess the functional effects of ICAD in respect of the prevention of the progression of cardiac insufficiency as an inhibitor of the key step in myocardial cell death, echocardiographic and haemodynamic parameters were measured following the adenovirus-mediated transcoronary gene transfer. Before implantation of the pacemaker, the rabbits were treated by means of gene transfer. Echocardiographic and haemodynamic parameters were recorded after a pacemaker time of 7 and 15 days (with the pacemaker switched off). Rabbits infected with Ad-GFP served as control hearts. FIG. 6a shows the mean values of the fractional shortening of the infected region for Ad-GFP and Ad-ICAD. A clear significant increase occurred in animals treated with Ad-ICAD, but no action occurred in the control animals treated with viruses (15±0.9% vs. 22.3±1.1%, n=6 in each group, p<0.05). The end-diastolic diameter of the left ventricle was 18±4 mm in the Ad-GFP animals, as compared with 15±0.5 mm in the ICAD-infected animals (FIG. 6b). In order to supplement the echocardiographic information regarding the local myocardial contractile force, the end-diastolic pressure of the left ventricle and +dp/dt were determined as a measure of the global ventricular contractile force. The end-diastolic pressure in the left ventricle of animals treated with adenoviruses for ICAD was restored to a significant extent again as compared with the Ad-GFP-infected cells (10.2±0.8 mm Hg vs. 14±1.4 mm Hg, n=6 in each group, p<0.05) (FIGS. 6c,d). The basal +dp/dt values of Ad-GFP-infected animals did not differ from those of Ad-ICAD-infected animals. After injection of adrenalin, a dose-dependent higher increase in respect of +dp/dt in the ICAD-expressing rabbits was observed as compared with the control animals.

### EXAMPLE 7

### The Functional Reconstitution of the Contractile Force is Achieved by a Reduced Worsening of the Sarcomere Organisation by Overexpression of p35 or ICAD

In order to study the effect of p35 expression on the contraction function of individual ventricular cardiac muscle cells 15 days after administration of the gene in vivo, cells were isolated from the target region of control animals infected with Ad-p35 and Ad-GFP and CHF animals and cultured for 18 hours. Cells expressing the transgene were selected by means of GFP fluorescence. FIGS. 8 (a,b,c) shows laser electron microscopic images after phalloidin staining for the visualisation of polymeric actin in control cardiac muscle cells and insufficient cardiac muscle cells, with had been isolated from genetically manipulated hearts. Interestingly, failing cardiac muscle cells expressing p35 exhibited a more highly organised sarcomere structure in comparison with control-infected cells having destroyed sarcomere units. The degree of sarcomere organisation, determined by semi-quantitative evaluation, is shown in FIG. 8(e). The ratio of muscle cells to well-organised sarcomeres (more than ⅔ of the cell region) was 64%±1% in control cardiac muscle cells, 13%±3% in insufficient Ad-GFP-infected cardiac muscle cells and 52%±4% in Ad-p35-infected cardiac muscle cells. The contraction amplitude was measured in insufficient cells and control cells which had been stimulated electrically at a rate of approximately 70 contractions per minute. The adenovirus infection did not alter the contraction characteristics of the cardiac muscle cells. As shown in FIG. 8d, failing muscle cells expressing p35 exhibited a significantly increased fractional shortening in comparison with equivalent muscle cells infected with Ad-GFP (4.3±0.4% vs. 1.8±0.2%, n=40 in each group). This improvement in the contractile force could also be observed after isoprenaline stimulation, and the EC₅₀ of the dose-response curve was displaced to higher values, which were similar to those of healthy cells (FIG. 8d). These results show that p35 restores sarcomere organisation and the contractibility of failing cardiac muscle cells is restored again as a result.

Furthermore, the contraction amplitude was measured in insufficient cells and control cells which had been stimulated electrically at a rate of approximately 70 contractions per minute. As shown in FIG. 10, failing cardiac muscle cells expressing ICAD exhibited a significantly increased fractional shortening in comparison with equivalent Ad-GFP-infected muscle cells after isoprenaline stimulation (improvement from 3.8±0.9% to 9.5±1.16%), while the basal values did not differ (1.8±0.33% vs. 1.75±0.33%). These results show that ICAD also restores the contractibility of failing cardiac muscle cells.

### EXAMPLE 8

### Microinjection of Activated Caspase-3 into Isolated Ventricular Cardiac Muscle Cells

In order to be able to study whether the activation of caspase-3 is sufficient for the induction of the worsening of sarcomere organisation, the activated enzyme was injected into the cytoplasm of healthy adult ventricular cardiac muscle cells. Positive cells (approximately 10 to 20%) were identified by means of FITC fluorescence. The morphological features of the cardiac muscle cells after microinjection of activated caspase-3 (4 ng/μl and 20 ng/μl) in comparison with control-injected cells are shown in FIGS. 9(a+b). Treatment of the muscle cells with caspase-3 (CPP-32) effected a rapid, concentration-dependent destruction of the smooth and striated muscle bundles (FIG. 9b, left- and right-hand image). Shortening experiments with individual cells (microinjected cardiac muscle cells) exhibited a caspase-3-mediated lowering of the basal and isoprenaline-stimulated contraction (9.8% vs. 4.3% [10⁻⁸ iso]; n=30 in each group). These effects were blocked by pre-incubation of the muscle cells with DEVD-fmk (FIG. 9c).

1. Use of a caspase-3 or CAD inhibitor or of a vector that comprises a nucleic acid sequence coding for the inhibitor, in the preparation of a medicament for improving the contractile force of the heart in the prevention or treatment of cardiac insufficiency, the inhibitor being selected from
(a) a ribozyme or an antisense RNA that inhibits the expression of caspase-3 or CAD;
(b) ICAD;
(c) Baculovirus p35;
(d) antibodies that bind specifically to caspase-3 or CAD, or a fragment thereof.

2. Use according to claim 1, wherein the cardiac insufficiency is insufficiency of the left ventricle.

3. Use according to either claim 1 or 2, wherein the prevention or treatment of cardiac insufficiency is so carried out that the DNA sequence coding for the inhibitor is administered inserted into an expression vector.

4. Use according to claim 3, wherein the vector is an adenovirus.

5. Screening process for identifying a compound which as a caspase-3 or CAD inhibitor is able to improve the contractile force of the heart in the prevention or treatment of cardiac insufficiency, the process comprising:
